(19) United States
(12) Patent Application Publication
(10) Pub. No.: US 2005/0165325 A1
(43) Pub. Date: Jul. 28, 2005

(54) Hospital bed

(75) Inventor: Mathias Hornig, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/042,862

(22) Filed: Jan. 25, 2005

(30) Foreign Application Priority Data
Jan. 26, 2004 (DE) 10 2004 003 717.5

(51) Int. Cl. A61B 5/00; A61B 5/02; A61B 5/08

(52) U.S. Cl. 600/549; 5/600; 600/484

(57) ABSTRACT

The invention relates to a hospital bed (1) with a support part (2) for a patient, said bed being provided with a moveable holder with a display (6) and/or with sensors (10 to 12) for recording measurement data. Measurement data of the patient can be recorded at all times by means of the sensors (10 to 12). These values can be displayed by means of the display (6). By means of a connection (9) to a multimedia network, these values can be communicated from the hospital bed. An input device (16) can be used to record patient data directly at the hospital bed.

### CROSS REFERENCE TO RELATED APPLICATIONS

This application claims priority to the German application No. 10 2004 003 717.5, filed Jan. 26, 2004 which is incorporated by reference herein in its entirety.

### FIELD OF INVENTION

The invention relates to a hospital bed with a support part for a patient. Hospital beds of this type have hitherto only been used in their function as a bed, even though patients spend a large part of their time in hospital in them.

### BACKGROUND OF INVENTION

Handwritten entry of patient data, such as the name, diagnosis measurement data etc, onto a form which is fixed to the foot of the bed is generally known. External devices are used for recording measurement data about the patient, said devices having to be taken to the bed and thereby impairing access to the patient. Communication is only possible in one direction from the patient to the nurses' office by means of a bell above the bed.

U.S. Pat. No. 4,768,241 discloses a hospital bed for an intensive care unit, whereby a monitor is provided for displaying patient-related measurement data, which is measured by the sensors attached to the hospital bed. For monitoring purposes, this measurement data can also be forwarded to a control centre remote from the hospital bed by means of connections provided therefor.

U.S. Pat. No. 5,970,980 discloses a holder for a video monitor for use in an operating theatre, said device making it easy for the surgeon to view video images taken by an endoscopic camera used during a surgical operation. One exemplary embodiment describes a longitudinally moveable holder arranged on a hospital bed.

### SUMMARY OF INVENTION

An object of the invention is to design a hospital bed of the type mentioned at the beginning, which is multifunctional and can also be used for the simple handling of patient data, measurement data as well as for communication. The object is achieved by the claims.

The display according to the invention of the patient data on the display which can be retrieved via the network connection enables the handwritten recording of patient data on a form and the fixing of said form to the foot of the hospital bed which would otherwise be necessary to be dispensed with. Handling the patient data in this way also enables said patient data to be displayed in a simple manner on a different bed, in the event of the patient being relocated to a different department.

Sensors are provided in one embodiment so as to use the hospital bed in an advantageous manner to record measurement data. The measurement data measured using these sensors can be displayed on the display and/or by means of the network connection, e.g. transmitted to a hospital information system or to a control centre.

The hospital bed can also be used for communication if it is connected to a multimedia network by means of a connection, and the display is a multidata display. By way of example, if the display is provided with a input device, in particular in the form of an alphanumeric keyboard, the patient data can be recorded directly at the hospital bed.

The input device is advantageously provided for inputting patient data. This patient data is transmitted to the hospital information system or to the control centre so that a centrally stored patient record can be processed at the hospital bed of the respective patient.

The hospital bed can advantageously be provided with sensors for recording measurement data, which determines the weight, body fat, body temperature and/or blood pressure of the patient.

According to the invention, the sensors can comprise an automatic pulse detection unit.

It has proved advantageous for a blood pressure cuff to be attached to a hospital bed with the aid of a roll-up mechanism.

The sensors can advantageously comprise an analysis unit for urine and/or stools.

The patient can be observed for example from the nurse's office, if the display features a camera.

The display can be used for television or video playback, if it is provided with loudspeakers.

Warnings or notifications can be issued to the patient if the display is provided with a signal lamp.

### BRIEF DESCRIPTION OF THE DRAWINGS

The invention is described below with reference to exemplary embodiments shown in the drawing, in which;

FIG. 1 shows a hospital bed with a configuration according to the invention,

FIG. 2 shows a view from above of the hospital bed with sensors according to the invention,

FIG. 3 shows a multidata display according to FIG. 1.

### DETAILED DESCRIPTION OF INVENTION

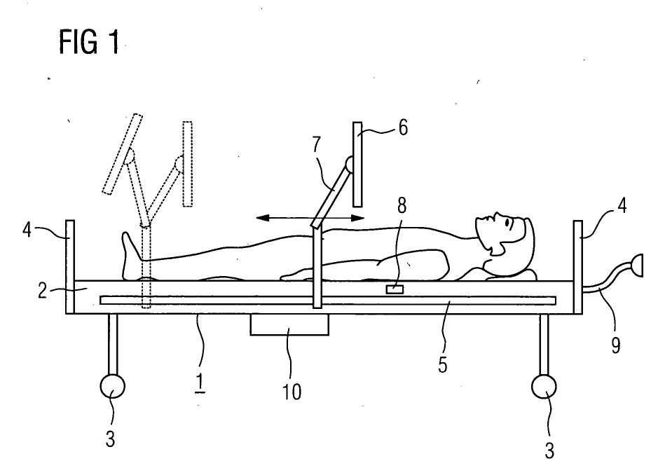

FIG. 1 shows a hospital bed 1 with a support part 2, which can be moved on castors 3. The support part 2 has a head and foot part 4. A track 5 for guiding a multidata display 6 is located on the side of the support part 2, with a swivel arm support arm allowing the display to be turned or tilted. The swivel arm 7 of the multidata display 6 can be controlled by an electric motor or manually adjusted.

A pad 8 for controlling the swivel arm 7 of the multidata display 6 is also attached to the side of the support part 2, so that both patient and staff can operate the multidata display 6. The hospital bed 1 is connected to a hospital information system (HIS) and to a control centre (nurses' office) by means of connection 9 through the multimedia network. The connection 9 can however also be made via radio. An analysis unit 10 for urine and/or stools is arranged below the support part 2.

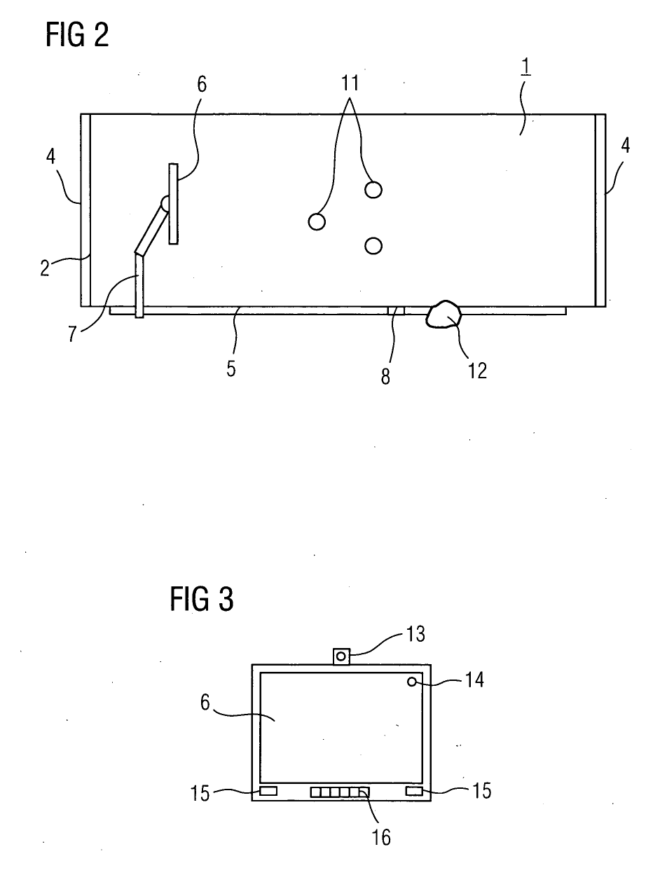

FIG. 2 shows a view from above of the support part 2 of the hospital bed 1. Sensors 11 are incorporated into the surface for recording the weight, body temperature, body fat etc. A cuff 12 for pulse measurement and blood pressure measurement is attached to the side of the hospital bed 1, with the aid of a roll-up mechanism (not shown) for recording the blood pressure and the pulse of the patient.

FIG. 3 shows the multidata display 6 which is provided with a camera 13. The camera 13 enables the patient to be monitored from the nurses' office for example. Communication with a staff nurse is also possible in this manner. Furthermore, the multidata display 6 comprises a signal lamp 14 as a warning and/or indicator lamp, by means of which said indications can be transmitted to the patient from the nurses' office.

The multidata display 6 can also be used for video or television playback, if it is provided with loudspeakers 15 (as shown). For this purpose, said display can be provided with its own receiver section, to which an antennae signal is supplied by means of connection 9, or a video signal of a receiver is conveyed to the multidata display 6. Furthermore, it can be used for displaying patient data which can be recalled from HIS by means of the connection 9. It can also be used as information medium, as a video telephone for example. Similarly the examination images can be displayed on the multidata display 6, which can be retrieved from an image archive by means of connection 9.

An input device 16 is attached to the multidata display 6, which can comprise individual keys by means of which specific functions can be selected, thereby enabling acknowledgements to the control centre. The input device 16 can also be an alphanumeric keyboard, by means of which patient data can be input and then transmitted into patient records by means of connection 9. The multidata display 6 can also be provided with a touch screen as an input device 16.

To summarize, according to the invention the hospital bed has the following features:
1. a network connection to the HIS (hospital information system) and to the control centre (nurses' office)
2. a data display with an input function (e.g. touchpad)
3. integrated scales with an analysis unit for body fat
4. an analysis unit for urine/stools
5. a blood pressure meter
6. an automatic pulse detection unit.

The networked, multifunctional clinical bed with integrated monitoring functions uses the effect of the long time spent in hospital and availability of the patient for information, data acquisition and communication. The staff effort involved in data acquisition is reduced, as are errors made when entering the data. The invention enables data to be recorded at regular intervals, regardless of personnel availability, especially during the night.

With the aid of the network connection 9, to the HIS and to the control centre (nurses' office) for example, the possibility exists of;
Online querying of patient data by the doctor,
Automatic acquisition and storage of patient data and monitoring data and
Automatic updates of information on the data display.

The data display with input function, a touchpad for instance, can be aligned flexibly to the doctor and the patient, in a similar manner to displays integrated in airplane seats. On the one hand, it can be used for visualizing patient data or images. When the patient checks into the hospital, data is sent to the display with the name and diagnosis for example. If the patient is relocated to another department, the data can be sent from the control centre to the new bed and updated. On the other hand, the data display can be equipped with information from the nurses about specific measures, such as visiting times, meal plans, and treatment schedules, but also visual and audible warning notifications, which is extremely useful in terms of efficiency and cost. For example, the patient can be audibly or visually informed about taking their medication and should confirm that they have taken it. If said patient fails to do this, the nurses are informed by way of the network connection.

Data for weight and fat ratio can be monitored by confining the patient until his/her discharge, using the integrated scales with an analysis unit for body fat. The scales with sensors for the body fat analysis are integrated into the surface of the support part 2. Data acquisition takes place automatically.

If the patient is not mobile and wets the bed for example, this can be removed from the basin for analysis. The analysis unit 10 for urine/stools is compact and is located under the support part 2. The results are assigned to the patient record by means of the network.

The blood pressure cuff 12 of the blood pressure meter is attached to the side edge of the bed of the support part 2, with the aid of a roll-up mechanism for example. The data is automatically recorded depending on the requirements and sent to the patient record by radio or by cable.

The data recorded by means of the automatic pulse detection unit, for example the blood pressure cuff 12, is recorded and processed in the same way as the blood pressure measurement.

1-17. (canceled)

18. A hospital bed, comprising:
a display holder;
a display device supported by the display holder; and
a computer network device for connecting the display device to a patient data database so that patient data pertinent to a specific patient can be displayed on the display device.

19. The hospital bed according to claim 18, further comprising a sensor for acquiring measurement data related to vital functions of the specific patient, wherein the measurement data are displayed on the display device or transmitted by the computer network device to a hospital information system or a control center.

20. The hospital bed according to claim 18, wherein the hospital bed is connected to a multimedia network using the computer network device and the display device is adapted to display a plurality of different data types.

21. The hospital bed according to claim 18, to 3, wherein the display device includes an input device.

22. The hospital bed according to claim 21, wherein the input device is an alphanumeric keyboard.

23. The hospital bed according to claim 22, wherein the input device is used for inputting at least part of the patient data, the patient data transmitted via the computer network device to a hospital information system or a control center.

24. The hospital bed according to claim 18, wherein the display holder is moveably arrange at a lying area of the hospital bed.

25. The hospital bed according to claims 18, wherein the display holder includes a swivel arm and the display device is supported by the swivel arm.

26. The hospital bed according to claim 25, wherein the swivel arm is adapted to turn and tilt the display device towards a patient lying in the hospital bed or towards a doctor standing adjacent to the hospital bed.

27. The hospital bed according to claim 19, wherein the sensor is a weight sensor for determining the weight of the specific patient.

28. The hospital bed according to claim 19, wherein the sensor is adapted to determine an adipose value of the specific patient.

29. The hospital bed according to claim 19, wherein the sensor is adapted to determine a blood heat of the specific patient.

30. The hospital bed according to claim 19, wherein the sensor is adapted to determine a blood pressure of the specific patient.

31. The hospital bed according to claim 19, wherein the sensor comprises a pulse detection unit.

32. The hospital bed according to claim 30, wherein the sensor includes a blood pressure cuff attached to the hospital bed using a roll-up mechanism.

33. The hospital bed according to claims 19, wherein the sensor comprises an analysis unit for acquiring measurement data related to urine or stool of the specific patient.

34. The hospital bed according to claim 18, wherein the display device includes a camera.

35. The hospital bed according to claims 18, wherein the display device includes a loudspeaker.

36. The hospital bed according to claim 18, wherein the display device includes a signal lamp.
